(19) United States
(12) Patent Application Publication
(10) Pub. No.: US 2016/0058821 A1
(43) Pub. Date: Mar. 3, 2016

(54) PLANT BASED FORMULATION FOR THE PREVENTION AND MANAGEMENT OF OBESITY AND ASSOCIATED COMPLICATIONS

(71) Applicant: SRM UNIVERSITY, Kattankulathur (IN)

(72) Inventors: Govind P. Dubey, Kattankulathur (IN); Aruna Agrawal, Varanasi (IN); Shipra Dubey, Kattankulathur (IN); Rajesh Dubey, Kattankulathur (IN)

(73) Assignee: SRM UNIVERSITY, Kattankulathur (IN)

(21) Appl. No.: 14/469,272

(22) Filed: Aug. 26, 2014

(51) Int. Cl. A61K 36/8945; A61K 36/38; A61K 36/185; A61K 36/37

(52) U.S. Cl. CPC A61K 36/8945; A61K 36/37; A61K 36/38; A61K 36/185

(57) ABSTRACT

The invention relates to a plant based formulation for the prevention and management of obesity and associated complications comprising of an effective amount of hydro-alcoholic extract of Salacia reticulata, Garcinia indica, Dioscorea bulbifera, Terminalia chebula and optionally additives in trace amounts.

### FIELD OF INVENTION

The present invention relates to a plant based formulation for the prevention and management of obesity and associated complications like hypertension, insulin resistance, dyslipidemia and coronary heart disease. The formulation is beneficial in managing particularly adipokines i.e. leptin, adiponectin including ghrelin and resistin.

### BACKGROUND OF THE INVENTION

Over weight and obesity poses a major public health challenge. Obesity is a chronic condition that develops as a result of an interaction between a person's genetic make up that are not well understood. However, social, behavioral, cultural, psychological, metabolic factors are also contributing to obesity. Obesity is the major health care challenge for the 21^{st} century, and its prevalence is increasing globally (WHO 1997, Popkin B M et al 1997). The prevalence of obesity and overweight is influenced by a complex interaction of genetic, environmental and behavioral factors. Obesity occurs when caloric intake exceeds than energy expenditure, but a growing body of evidence supports the view that obesity is caused by inherited tendencies to subtle disorders of the weight regulating mechanism magnified by a poor diet and lack of activity practiced by most of the population. (Mokdad. A. et al., 2004, N.I.H., 2004). Unfortunately, there has been a phenomenal increase in the prevalence of obesity in India, particularly in the urban area. Obesity is risk factor for a wide range of disease and is associated with increased morbidity and reduced life expectancy. Several studies reported childhood and adolescence obesity is associated with pre-matured death in adulthood (Rosengren A et al 1999, Yarnell J W et al 2000, Engeland A 2003). Epidemiologic studies have shown a strong correlation between socio-economic status and prevalence of obesity. This relationship is stronger in women (Calle E E et al 1999, Bender R et al 1999).

Overweight and obesity have reached epidemic proportion in U.S.A, increasing among all age, race and ethnic groups and in both men and women. The most recent survey from National Health Examination Survey (NHANES) for 1999 to 2000 reported the age-adjusted prevalence of overweight [defined as BMI: 25 kg/m²] as 64.5% and obesity [defined as BMI: 30 kg/m²] as 30.5° A for U.S. adults (Flagal, K. M. et al., 2002). The prevalence of obesity and overweight continues to increase dramatically, in past, as an intended consequence of the economic, social and technological advances that have contributed to unhealthy dietary habits and sedentary behavior during the past several decades. It is the second leading cause of preventable death in the U.S. today accounting for ˜300,000 deaths per year (Allison, D. B et al; 1999).

Recently, it is reported that the prevalence of obesity is markedly increased i.e. 19.3 percent in 2001 to 22.7 percent in 2007. Similarly there is continuous increase in the prevalence of diabetes and obesity causing serious medical complications. As obesity is a significant risk factor for development of diabetes therefore a drastic preventive measures are required for the obesity to prevent future development of insulin resistance. A socio-cultural factor is associated with prevalence of obesity and diabetes. Very recently Allison L. et al (2010) reported that high prevalence is associated with lower income and comparatively least educated group of people.

Several evidences indicate that obesity is associated with an increased risk of cardiovascular disease, type-II diabetes mellitus, hypertension, stroke and dyslipidemia (Huang Z et al 1997, Shoff S M et al 1998). Joint national committee on prevention, evaluation and treatment of high blood pressure (1997), defined obesity as the major risk factor for CVD and associated mortality. Numerous studies showed that the pattern of body fat distribution affects morbidity and mortality from cardiovascular disease (Bouchard C et al 1990, Lapidus L et al 1984). Many studies have demonstrated the beneficial effect of weight reduction on cardiovascular risk factors such as hypertension and dyslipidemia (Scherrer V et al 1991, De Simone A et al 1992, Rossner S et al 1987).

Overweight and obese people have an increased incidence of cardiovascular disease (CVD), including heart attack, congestive heart failure, sudden cardiac death, angina and abnormal heart rhythm (office of the U.S. Surgeon General, 2004).

Excess fat and weight is combined with adverse metabolic changes associated with chronic degenerative diseases that increase both morbidity and mortality. Obesity has a negative effect on lipid levels in the blood, which often leads to the development of a condition known as dyslipidemia. Dyslipidemia, a primary risk factor for coronary artery disease, occurs when LDL cholesterol and triglyceride levels are high, HDL cholesterol is low or any combination of these factors. People with an excessive amount of body fat have higher levels of TG and LDL and lower level of HDL cholesterol in the blood which may cause inflammation and an increased risk of developing CVD. (N.I.H., 2004). Physicians often attribute this abnormal shift in lipid levels to weight gain. Losing weight conversely has an opposite effect.

A high level of cholesterol in blood is associated with an increased risk of heart attack because cholesterol is major component of the plaques deposited in arterial walls. Cholesterol, like oil, cannot dissolve in blood unless it is combined with special proteins called lipoproteins. The cholesterol combined with LDL is “bad” cholesterol that deposits cholesterol is associated with an increased risk of heart attack (Archives of Internal Medicine, 2000).

High blood pressure is a risk factor for developing atherosclerosis and heart attack. Both high systolic pressure and high diastolic pressure increase the risk of heart attack.

More than 80% people with the most common form of type-2 diabetes are obese or overweight. Data from CDC's National Health and Nutrition Examination Survey III shows that ⅔^{rd} of adult men and women in the U.S. diagnosed with type 2 diabetes have a BMI of 27 or greater, which is classified as overweight and unhealthy (Archives of Internal Medicine, 2000).

As obesity alters the body's ability to control blood sugar using insulin, there is an increased risk of developing diabetes. The body begins over producing insulin to regulate blood sugar levels but over time the body can no longer keep these levels in a normal range. Eventually this inability to achieve balance results in higher, unhealthy blood sugar levels, ultimately leads to the development of type 2 diabetes (North American Association for study of obesity, 2004). Obesity complicates the management of type 2 diabetes by increased insulin resistance and glucose intolerance, which makes drug treatment for the disease less effective (American Obesity Association, 2004).

Hs C-reactive protein (CRP) is correlated with pre-abdominal obesity and a raised level predicts the risk of developing type-II diabetes (Freeman D J et al 2002, Barzilay J I et al 2001, Pradhan A D et al 2001). Obesity mediated cytokine production is another important central mechanism for systemic elevation of CRP and IL-6. A strong correlation with markers of endothelial activation and dysfunction is reported (Yudkin J S et al 2000). It is reported that IL-6 and CRP levels are responsible for adipocyte activation. Other potential mediators of insulin resistance deriving from adipose store is TNF a (Hotamisligil G S et al 1995, 1996), leptin (McNeely M J et al 1999), free fatty acid (Groop L C et al 1991) and resistin (Steppan C M et al 2001). The identification of obese and non-obese individuals and risk for the development of diabetes the measurement of above biomarkers are significantly useful.

TNF-α decreases tyrosine kinase activity of the insulin receptor and is overproduced in adipose tissues in insulin-resistant rodents and humans suggesting that it is a possible mediator of insulin resistance in obesity and diabetes (Hotamisligil G S et al 1993, 1994, 1994a).

Obesity results in marked alterations in cardiac functions due to increased fatty acids uptake and oxidation. Obesity also causes release of adipokines, such as leptin and adiponectine which have shown significant impact in the regulation of cardiac functions. There is association between obesity, cardiovascular disease, lipid metabolism and adipokines. Human obesity is characterized by elevated plasma leptin levels and resistance to the metabolic effects of the hormone to the extent that high plasma leptin levels are ineffective in reducing fat accumulation (Narkiewicz K et al 1999). In humans, there is evidence of leptin resistance and leptin concentration increases with obesity and correlates strongly with percentage body fat in both sexes (Mohamed Ail et al 1998). Similarly, one of the epidemiological studies of 2537 men and women showed a strong association between fasting insulin level, leptin concentration and obesity (Zimmet P Z et al 1998). Further, higher leptin levels are also associated with the development of obesity and type-II diabetes mellitus (Chessler S D et al 1998, McNeely M J et al 1999).

Obesity related disorders including metabolic syndrome, diabetes, atherosclerosis, hypertension and coronary artery disease are associated with decreased plasma levels of adiponectin, insulin resistance and endothelial dysfunction (Han S H et al 2007). Its levels are low in obesity and increases after weight loss (Arita Y et al 1999, Weyer C et al 2002, Yang W S et al 2001).

Treatment of obesity should be based on its severity. Despite the magnitude of the problem of obesity, most health care provides uncomfortable dealing with it. This has led to publication of a set of guidelines on the treatment of obesity by N.I.H. urging health care provides to address obesity in the office setting (Aronne L J 2002).

Sibutramin and Orlistat both the drug are approved for obesity management by the FDA (Rolls B J et al 1998, Hill J O et al 1999), but its long term application are not safe and causes mood enhancement and cardiovascular excitation (Graddoc K D 1978, Abenhain L et al 1996, Bowen R et al 1997). Recent data suggested that the statin therapy based on CRP values would result in fewer events and regression of atherosclerosis. Elevation of CRP is related to obesity, diabetes, estrogen therapy, hypertension smoking etc.

The classical literature of Ayurveda has given a comprehensive description about the etiopathogenesis and management of obesity (ati-sthula or medo-roga) (Ashtau-ninditiyādhyaya, Charak sutra 21:4-10). Charak has described the genetic (Sahaj) and environmental factors responsible for excessive deposition of meda-dhātu. The genetic pre-disposition is one of the most important factors of obesity.

Recently maximum time and attention has been paid towards the utilization of plant based formulations useful in the prevention and management of various clinical conditions as these agents have shown better efficacy over standard conventional pharmacologic therapies and found to be more safer side as such drugs have no adverse effects on various body system. Thus based on ancient medical literature lead was taken and a novel plant based formulation was evaluated following multi-targeted anti-obesity activity among obese/over weight patients.

### OBJECTS OF INVENTION

The primary object of present invention is to propose a plant based formulation effective in reducing high body mass index (BMI) including hip waist ratio (WHR), reducing body weight among the obese patients.

Further object is to propose a plant based formulation beneficial in the prevention and management of insulin resistance among obese individuals.

Another object of present invention is to propose a plant based formulation beneficial in the management of atherogenic dyslipidemia by reducing elevated low density lipoprotein cholesterol and triglycerides among obese cases.

Yet another object of present invention is to propose a herbal formulation effective in reducing pro-inflammatory cytokines i.e. TNF-α and IL-6 to prevent obese individuals from vascular complications associated with obesity.

Still, another object of present invention is to propose a plant based formulation effective in the prevention from low grade inflammation among obese individuals.

Another object of present invention is to prevent obese patients from development of diabetes by preventing insulin resistance among obese cases.

The ultimate object of present invention is to propose a plant based formulation beneficial in preventing the obese patients to develop coronary heart disease.

The foregoing has outlined some of the pertinent objectives of the invention. These objectives should not be construed to be merely illustrative of some of the more prominent features and applications of the intended invention. Many other beneficial results can be obtained by applying the disclosed invention in a different manner or modifying the invention within the scope of disclosure.

Accordingly, other objectives and a full understanding of the invention and the detailed description of the preferred embodiment in addition to the scope of invention are to be defined by the claims undertaken.

These and other objects and advantages of the invention will be apparent from the ensuing description.

### BRIEF DESCRIPTION OF DRAWINGS

Further objectives and advantages of this invention will be more apparent from the ensuing description when read in conjunction with the accompanying drawings wherein:

FIG. 1(a), FIG. 1(b), FIG. 1(c). Flow Diagram of the process.

FIG. 2. Effect of test formulation on body mass index in obese Cases.

FIG. 3. Effect of test formulation on obesity index measured in terms of Tricep Skin fold thickness in obesity cases

FIG. 4. Effect of test formulation on obesity index measured in terms of Sub-scapular Tricep Skin fold thickness in obesity cases.

FIG. 5. Effect of test formulation on Leptin levels among obese cases.

FIG. 6. Effect of test formulation on Adfiponectin levels among obese cases.

FIG. 7. Decrease in Triglycerides level following test drug treatment among obese cases.

FIG. 8. Reduction in Endotheline following drug treatment among obese cases.

FIG. 9. Reduction in Apolipo content following drug treatment among obese cases.

While the invention is described in conjunction with the illustrated embodiment, it is understood that it is not intended to limit the invention to such embodiment. On the contrary, it is intended to cover all alternatives, modifications and equivalents as may be included within the spirit and scope of the invention disclosure as defined by the claims.

### STATEMENT OF THE INVENTION

According to this invention, there is provided a plant based formulation for the prevention and management of obesity and associated complications comprising of an effective amount of hydro-alcoholic extract of Salacia reticulata, Garcinia indica, Dioscorea bulbifera, Terminalia chebula and optionally additives in trace amounts.

In other embodiments, the invention is a plant based formulation comprising at least two hydro-alcoholic extracts selected from Salacia reticulata, Garcinia indica, Dioscorea bulbifera, and Terminalia chebula. Exemplary formulations include hydro-alcoholics extracts of Salacia reticulate and Dioscorea bulbifera; hydro-alcoholic extract of Garcinia indica, Dioscorea bulbifera, and Terminalia chebula; hydro-alcoholic extract of Salacia reticulate, Garcinia indica, and Terminalia chebula; hydro-alcoholic extract of Salacia reticulate, Dioscorea bulbifera, and Terminalia chebula; and hydro-alcoholic extract of Salacia reticulate, Garcinia indica, Dioscorea bulbifera, and Terminalia chebula.

### DETAILED DESCRIPTION OF THE INVENTION

At the outset of the description, which follows, it is to be understood that the ensuing description only illustrate a particular form of the invention. However, such a particular form is only an exemplary embodiment and the teachings of the invention are not intended to be taken restrictively.

For the purpose of promoting an understanding of the principles of the invention, reference is now to be made to the embodiments illustrates and the specific language would be used to describe the same. It is nevertheless to be understood that no limitations of the scope of the invention is hereby intended, such alterations and further modifications in the illustrated bag and such further applications of the principles of the invention as illustrated therein being contemplated as would normally occur to one skilled in the art to which the invention relates.

The hydro-alcoholic extract of four medicinal plants Salacia reticulata, Dioscoria bulbifera, Terminalia chebula and Garcinia indica is used and extraction was done units specific part of the plant, extracted in 50:50 ratio of aqueous and alcohol, the quantity in percent of active compound was determined and the extract was used for experimental and clinical studies. The water used to obtain extractive material of the plants and to prevent from any abnormal growth the reverse osmosis plant set was used. After extraction the active molecule was identified, separated and quantified through HPTLC and NMR methods.

A mechanism based studies were conducted to evaluate the mode of action of single plant extract as well as combined formulation. The anti obesity, anti-atherosclerotic, anti-inflammatory and leptin reducing as well as adiponetin regulating activity was proven by designing specific animal model of obesity. The test formulation exerted its beneficial role on various bio-markers by acting on cholesterol receptors, showing activity on adiponectin gene expression and leptin reducing property, improved insulin resistance and reducing vascular inflammation by reducing interleukin-6 (IL-6) and tumor necrosis factor-alpha (TNF-α). On the whole it was proven that test formulation has a strong anti-obesity role thus showing its potentiality in prevention and management of obesity and associated complications which prevents the development of coronary involvement among the obese patients.

### Extraction Procedure

Four medicinal plants as described above were collected and their parts used for medicine purposes were shade dried and extracted as per procedure mentioned above. Like wise the root of Salacia reticulata, rhizomes of Dioscorea bulbifera, fruits and seeds of Terminalia chebula, and fruit of Garcinia indica were separately used for obtaining extracted materials. The temperature for extraction was maintained at 60-70° C. and the pH of solution was also maintained at 6-8.

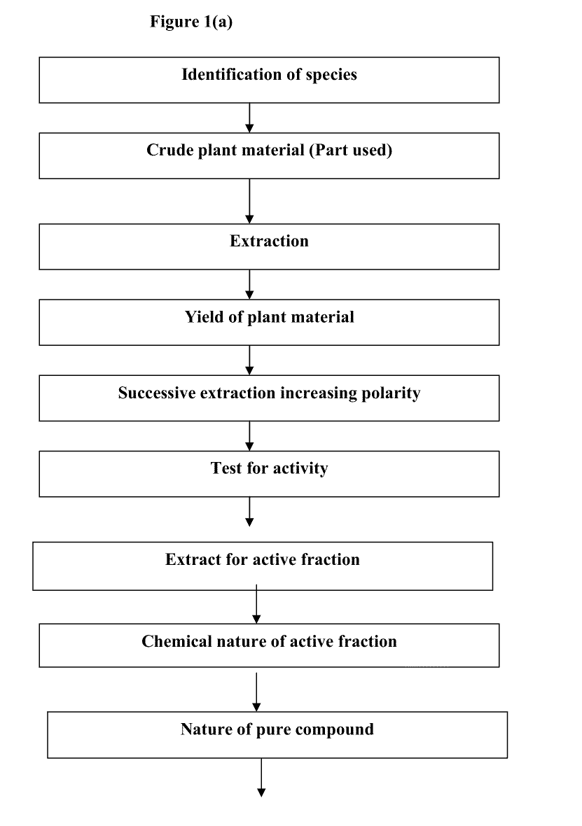

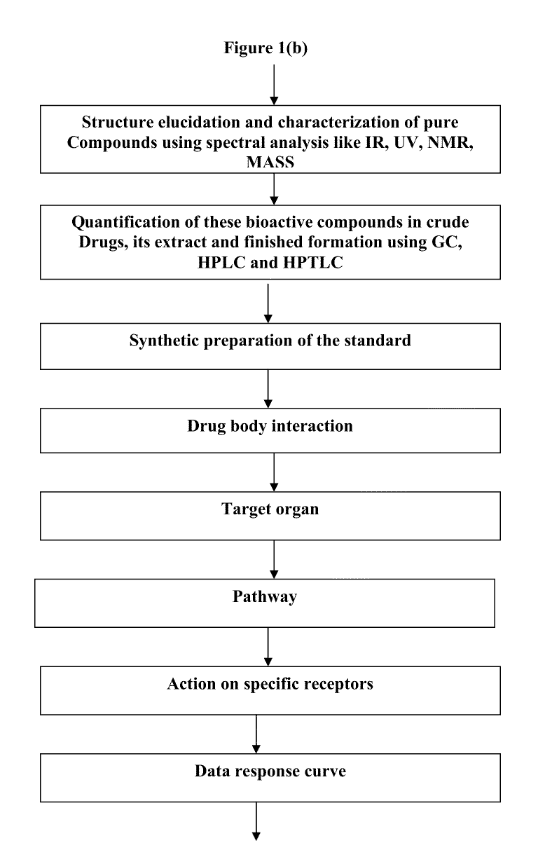

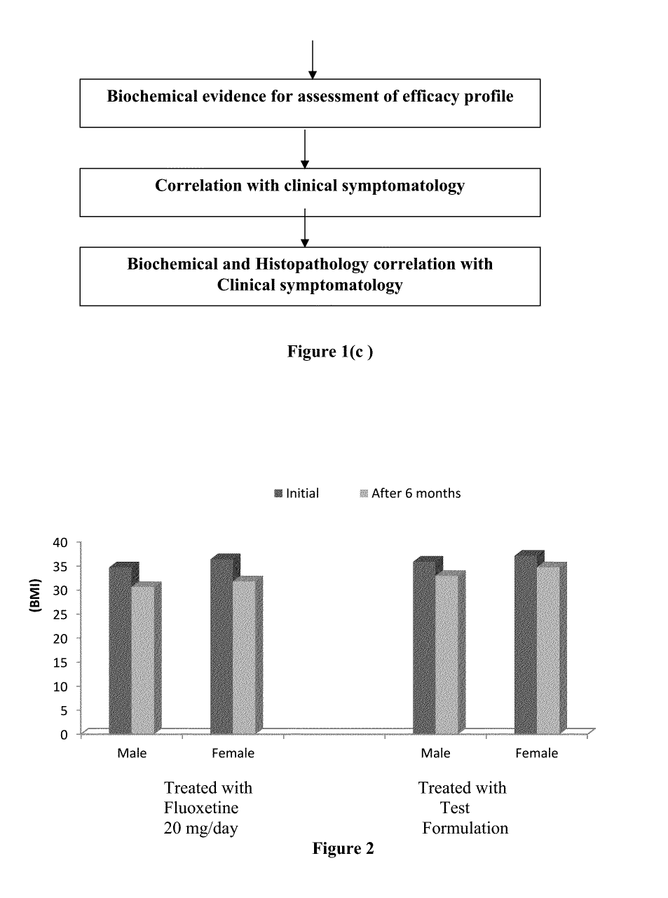

The steps, as listed in FIG. 1(a), FIG. 1(b) and FIG. 1 (c), were adopted to isolate the active compounds to assess the biological activity of test formulation evaluated on international guidelines prescribed for a new investigational drug entity.

According to present invention there is provided a plant based formulation showing beneficial role in the prevention and management of obesity and associated complications through its multi-targeted activity on various biochemical markers involved in the etio-pathogenesis of obesity. The present test formulation comprising of the following plant ingredients—

| Name of the plants | Part used |
|---|---|
| 1. Salacia reticulata | Root |
| 2. Garcinia indica | Fruit |
| 3. Dioscorea bulbifera | Rhizome |
| 4. Terminalia chebula | Fruits and Seeds |

Preferably the aforesaid plant extracts are present in the following dose range in test formulation—

| Name of the plants | Dose |
|---|---|
| 1. Salacia reticulata | 200-450 mg/day |
| 2. Garcinia indica | 100-300 mg/day |
| 3. Dioscorea bulbifera | 150-425 mg/day |
| 4. Terminalia chebula | 150-350 mg/day |

The formulation may also comprise known additives such as minerals, vitamins, salts, tiller (for capsulation or to prepare group) and binders if required to be present in trace amount.

Thus any known additive or supplement is added to prepare final capsule (formulation) as required for the purpose. Present test formulation is prepared in capsule form (500 mg each), however it may also be prepared in syrup/tablet form.

Preferably but without implying any limitation the present plant based formulation comprises—

| Name of the plants | Dose |
|---|---|
| 1. Salacia reticulata | 325 mg/day |
| 2. Garcinia indica | 150 mg/day |
| 3. Dioscorea bulbifera | 300 mg/day |
| 4. Terminalia chebula | 175 mg/day |

### Hypothesis

Recently it has been reported that obesity and insulin resistance is mainly characterized by high leptin and low adiponectin concentrations and is the basic markers for onset of obesity. Hypoadiponectinemia contribute to a low grade systemic chronic inflammatory state whereas insulin resistance is also significantly associated with increasing body weight and is a major risk factor for future onset of diabetes mellitus among obese patients. The effect of adiponectin diminution is also responsible for glucose intolerance as well as abnormal lipid metabolism. Therefore the present test formulation contains the extracts of Salacia reticulata, Garcinia indica, Dioscorea bulbifera, Terminalia chebula in effective doses as all these four plants have shown weight reducing, anti-inflammatory, anti-hyperlipidemic and insulin sensitivity enhancing activity. The test formulation has capability to reduce leptin level and enhances the adiponectin concentration as proven through experimental and clinical trials. In view of the above facts it is proposed that present test formulation has potential role in the prevention and management of major coronary risk factors particularly obesity thus it can prevent the future adverse cardiac event among the obese individuals. Further by control of body weight the complication like hypertension, osteoporosis, neurodegenerative disorders etc. can also be prevented. Thus it is proposed that since the obesity is a multi-factorial disorder and highly contributing in the development of coronary heart disease/ischemic heart disease and is the major cause for morbidity and mortality due to over weight and high BMI, the present test formulation contained the extract of four medicinal plants i.e. Salacia reticulata, Garcinia indica, Dioscorea bulbifera, Terminalia chebula. The plant Salacia reticulata, contains the active compound salacinol and kotalanol which has shown strong action on glucose metabolism and improves insulin sensitivity. It is a potent anti-obesity drug proven in several studies. Garcinia indica has been proven for its anti-oxidant, anti-inflammatory, lipase inhibitor (anti-obesity) as well as neuroprotective actions. Dioscorea bulbifera is the plant which has shown anti-hypercholesterolemic and triglycerides reducing activity and beneficial in the prevention and management of conventional coronary risk factors. The pharmacological action of Terminalia chebula is reported as blood glucose lowering, immunomodulatory, nephro-protective properties. In the light of these pharmacological therapeutic activity of plant candidates included in the present test formulation the present invention is of clinical significance for the management of obesity and obesity associated complications.

### About and Plant

1. Salacia reticulata (Saptachakra)

Salacia reticulata: Salacia reticulata belongs to Hippocrateaceae family. It is mainly distributed in Southern India. It is a large genus of climbing or creeping shrub or rarely small tree. Root and stem of Salacia reticulata are used for the treatment of diabetes mellitus in Ayurveda and Siddha System of Medicine. Salacinol, Kotalanol and kotalagenin 16-acetate showed a stronger inhibition of the increased serum glucose levels in maltose and sucrose loaded rats than acarbose (Matsuda et al 1999; Matsuda et al 2002). Salacia reticulata strongly inhibited the activities of α-glucosidase and α-amyalse, but not that of 3-glucosidase. (Shimoda et al 1998). In Siddha System of Medicine, Salacia reticulata has been used for the treatment of Diabetes mellitus. It is also useful in the treatment of inflammations, wounds, ulcers, liver diseases etc.

2. Garcinia indica (Kokum):

Belongs to the family Clusiaceae. Seeds and fruits of Garcinia indica is used for medicines and cosmetics. Hydroxycitric acid (HCA) is extracted from the rind of the fruit is used as hypocholesterolemic agent. It is also a potential anti-obesity agent. The anti-oxidant property of aqueous extract of the plant is observed. Garcinia indica extract is having anti-allergic effects. It contains oxalicacid, malic acid, polyphenols, carbohydrates, anthocyanin and ascorbic acid. Anti-oxidant property (Mishra A et al 2006) is due to Garcinol. Isogarcinol have shown anti-inflammatory activity, lipase inhibitor (anti-obesity) (Jena B S et al 2002) and neuroprotective effect.

Aqueous and ethanolic extract of Garcinia indica fruit-rind has shown ulcer protective activity against indomethacin induced ulcerogenesis and HCl/ethanol induced gastric lesion (Amol Bhalchandra Deose et al 2011). Water extract of Garcinia indica has shown anti-microbial properties against certain microbes and cytotoxic properties on Balb/C3T3 mouse fibroblasts (KN Varalakshmi et al 2010). In an experimental study on Wistar albino rats Garcinia indica Linn. Fruit rind aqueous and ethanolic extract has shown anti-oxidant and hepatoprotective effect (Amol Bhalchandra Deore et al 2011). Garcinol a polyisoprenglated benzophenane derivative has a potent anti-oxidant and a glycation inhibitor under specified conditions (Fumio yamaguchi et al 2000).

Garcinia indica fruit extract prevents cyclophosphamide induced oxidative stress in rat's brain by reducing power and free scavenging ability and higher phenol content (Tushrendra Singh et al 2011). In a Randomized clinical trials (RCTs) suggest that Garcinia indica extracts/HCA can cause short term weight loss the magnitude of the effect is small (Ighoonakpoya et al 2011). Treatment with Garcinia indica fruit extract could prevent cyclophosphamide induced oxidative stress in brain and it is a better protectant (Tushrendra Singh et al 2011).

3. Dioscorea bulbifera (Varahikand)

It is a member of the Dioscoraceae family. Tubers of Dioscorea have been used as a food and herbal medicine. The pharmacologically active components of Dioscorea species is diosgenin, which is a steroidal saponin and dioscin, a form of diosgenin with sugar attached. Recent studies suggest that dietary diosgenin may lower plasma cholesterol levels, reduce blood sugar and decrease inflammation.

In numerous studies, plant steroids have shown to lower plasma cholesterol levels in animal models. Dietary diosgenin has shown decrease in plasma cholesterol levels through reduced cholesterol absorption by the liver (Sauvaire Y et al 1991). Dubey G P et al (2008) studied the beneficial role of test formulation containing extract of Dioscorea bulbifera in the management of various CHD risk factors involved with menopausal women.

### Chemical Constituents:

Albuminoides, Fats, Carbohydrates, Furanoid norditerpenes—Diosbulbins A,B,C,D,E,F,G,H; Dihydrophenenthrene, tetrahydrophenenthrene, D-Sorbitol, A dihydrophenenthrene, 2,4,6,7-tetrahydroxy, 9,10 dihydroxyphenenthrene, phytoestrogen.

Diosgenin, a steroid sapogenin, is the product of hydrolysis by acids, strong bases, or enzymes of saponins, extracted from the tubers of Dioscorea wild yam, such as the Kokoro. The sugar-free (aglycone), diosgenin is used for the commercial synthesis of cortisone, pregnenolone, progesterone, and other steroid products and has shown anti-obesity, anti-inflammatory and hypolipidemic activity.

4. Terminalia chebula (Harilaki):

It belongs to family combrataceae and found all over India. Fruit and seed part is used for medicinal purpose which contains tannin, chebulic acid, chebulinic acid, ellagic acid, gallic acid and resin and used mainly for heart diseases. Studies have reported that Terminalia chebula protect the cells against free radical scavenging activities Terminalia chebula has shown anti-inflammatory, immunomodulatory, anti-diabetic and nephroprotective properties. The seed of this plant has shown anti-diabetic property proven in streptozotocin induced diabetes rat model.

Recently, Mahesh et al (2009) evaluated the anti-oxidant activity of aqueous extract of Terminalia chebula which protects the liver and kidney among aged rats. Reddy et al (2009) identified the novel compounds isolated from Terminalia chebula, are having the property to inhibit the COX and 5-LOX, the key enzymes involved in inflammation and carcinogenesis. In reference to metabolic syndrome, Singh et al., 2009 reported the significant effect of the fruit extract to control glucose lowering activity and to improve insulin sensitivity in animal models of type-2 diabetes mellitus.

### Chemical Composition:

Fruits contain astringent substances—Tannic acid, Chebulinic acid, gallic acid etc. Resin and a purgative principle of the nature of anthraquinone and sennoside are also present. Anthaquinone glycoside, chebulic acid, tetrachebulin, linoleic acid palmitic acid are the chemical constituents found in the fruits of the plants. Tannic acid is a polymer of gallic acid molecules and glucose. Tannic acid will hydrolyze into glucose and gallic or ellagic acid units. The anti-oxidant and anti-mutagenic properties of tannic acid are beneficial.

### Example-I

When the hydro-alcoholic extracts of Salacia reticulata in the dose of 30 mg/kg, Garcinia indica 15 mg/kg, Dioscorea bulbifera in the dose of 25 mg/kg and Terminalia chebula in the dose of 22 mg/kg body weight was combined and given to the (high fat diet model) experimentally induced obese rats showed body weight control, lipid lowering effects as well as serum insulin regulating role. The test formulation decreased the leptin level and increased the adiponectin concentrations following 3 months of treatment with formulation in comparison to control group.

### Example-II

The hydro alcoholic extract of plant Salacia reticulata in the dose of 100 mg/kg and Dioscorea bulbifera in the dose of 50 mg/kg body weight in combined from exerted reduction in body weight and abnormal lipids, lipoprotein lowering activity along with improved systemic inflammation as measured through hsCRP, interleukin₆ and TNF-α estimation.

### Example-III

In clinical trial program the test formulation revealed beneficial role on body weight control when the extract of Salacia reticulata (350 mg/day) and Dioscorea bulbifera (350 mg/day) was given to obese patients for 3 months. As it has shown there was a marked reduction in body mass index, skinfold thickness of various body parts without any adverse reaction.

### Example-IV

When the hydro-alcoholic extract of Terminalia chebula in the dose of 250 mg/day, Garcinia indica in the dose of 250 mg/day and Dioscorea bulbifera in the dose of 300 mg/day was administered to obese patients reduction in interleukin-6 and TNF-α was noticed suggesting anti-inflammatory effects. Further, this combination of three plant extract also exerted improved insulin sensitivity, reduction in blood glucose level among obese subjects.

### Example-V

When the hydro-alcoholic extract of Salacia reticulata in the dose of 450 mg/day, Terminalia chebula in the dose of 275 mg/day and Garcinia indica in the dose of 200 mg/day combined and given to selected obese patients for six months reduction in body weight along with reduced leptin and increase in adiponectin level was noticed suggesting the anti-obesity and drastic weight control role and improvement in atherosclerotic process following test formulation treatment.

### Example-VI

When the hydro-alcoholic extract of Dioscorea bulbifera in the dose of 400 mg/day, Terminalia chebula in the dose of 275 mg/day and Garcinia indica in the dose of 300 mg/day was given in combined form revealed marked reduction in total cholesterol, LDL-C and triglycerides and also increase in HDL-c indicating anti-hyperlipidemic activity of test formulation.

### Example-VII

When the hydro alcoholic extract of plant Dioscorea bulbifera in the dose of 350 mg/day, Terminalia chebula in the dose of 300 mg/day and Salacia reticulata in the dose of 375 mg/day to selected obese patients a significant improvement joint pain and work performance was noticed due to significant reduction in body weight and improved mental performance including feeling of well being, anxiety and depression of obese subjects.

### Example-VIII

When the hydro-alcoholic extract of all four plants i.e. Salacia reticulata in the dose of 325 mg/day, Garcinia indica in the dose of 150 mg/day, Discoria bulbifera in the dose of 300 mg/day and Terminalia chebula in the dose of 175 mg/day administered orally in combined form exerted maximum beneficial effect as there was reduction in body weight, total cholesterol, LDL-c, triglycerides levels and increase in HDL-c was noticed. The test formulation also reduced the increased interleukin-6 level, leptin level and increased the adiponectin concentration among treated obese individuals.

The pre-clinical and clinical studies conducted by us suggested that test formulation is safe and can be given for longer time.

It is claimed that better results are obtained when the extract of all four plants i.e. Dioscorea bulbifera, Salacia reticulata, Terminalia chebula and Garcinia indica were mixed and given in combined form in specific doses, is a potent anti-obesity, anti-inflammatory, anti-hyperlipidemic drug including anti-atherosclerotic actions and thus test formulation has potential role in the regulation of bio-markers associated with obesity and involved with CHD risk. Thus the test formulation reduces body weight by 10-15 percent, controls the increase in body weight, reduces blood pressure, improves insulin sensitivity, reduces abnormal lipids and lipoproteins and ultimately prevents from coronary heart disease among obese patients. The test formulation is safe, can be administered for longer time among human subjects.

### Experimental Evidence

### Pre-Clinical Efficacy Study of the Test Formulation

This experimental study was carried out to study the effect of test formulation was evaluated on the high fat diet induced obesity followed by hypercholesterolemia, vascular inflammation and other associated complications, with the object to develop a new plant based formulation beneficial in the prevention an management of obesity and its future consequences.
High fat diet (24.72% fat) induced obesity and effect of test formulation
Animals: Sprague dawley rats (both male and female)
For this study Sprague Dawley rats of both sexes were procured from central animal house, IMS, BHU.
Ethical approval was sought from Institutional Animal Ethics Committee. Rats were maintained under controlled condition (25° C., 12-hour light/dark cycle, 50% humidity) and fed either a standard chow diet or high fat diet (24.72% fat). The high fat palleted obesogenic diet was administered by ad-libitum access for six months. Study was divided in to three groups—
Group-I 10 Animals were Fed standard diet
Group-II 10 Animals were fed with high fat diet
Group-III 10 Animals were fed with high fat diet and treated with test formulation
   High Fat Diet Leads to—

Obesity, dyslipidemia, Insulin resistance, Increased systolic blood pressure, Endothelial dysfunction, glomerular nephritis, Decrease plasma adiponectin, Free Fatty acid.

### Composition for Normal and High Fat Diet

| Components | Normal Diet (%) | High Fat Diet (%) |
|---|---|---|
| Moisture | 7.36 | 3.24 |
| Crude protein | 16.45 | 22.15 |
| Crude Fat | 2.89 | 24.72 |
| Crude fiber | 12.24 | 3.6 |
| Calcium | 1.26 | 1.16 |
| Phosphorus | 0.85 | 0.86 |
| Total ash | 6.79 | 6.83 |

### Study Groups

| Group | | | Number of |
|---|---|---|---|
| No. | Treatment | Sex | Animals |
| I | Fed with standard diet | Male | 5 |
| | | Female | 5 |
| II | Fed with high fat diet (24.27% | Male | 5 |
| | fat) | Female | 5 |
| III | Fed High fat diet (24.27%) + | Male | 5 |
| | Test formulation | Female | 5 |

### Effect of High Fat Diet on Body Weight and Role of Test Formulation

| | | | | | | Average |
|---|---|---|---|---|---|---|
| | | | | | | Difference |
| | | Body Weight (g) | | | | Initial vs. |
| | | | After 1 | After 3 | After 6 | After 6 |
| Groups | Sex | Initial | month | months | months | months |
| Normal | Male | 258.92 ± | 266.88 ± | 271.52 ± | 286.93 ± | 28.01 |
| control | | 18.75 | 12.95 | 13.98 | 14.78 | |
| | Female | 234.85 ± | 241.92 ± | 252.26 ± | 259.29 ± | 24.44 |
| | | 10.14 | 13.45 | 11.04 | 8.95 | |
| High fat diet | Male | 241.82 ± | 269.98 ± | 294.42 ± | 335.90 ± | 94.08 |
| (HFD) | | 9.01 | 13.45 | 14.05 | 12.93 | |
| | Female | 238.46 ± | 253.85 ± | 291.48 ± | 344.10 ± | 105.64 |
| | | 10.98 | 13.11 | 12.82 | 8.91 | |
| High fat diet + | Male | 241.23 ± | 252.88 ± | 271.05 ± | 294.01 ± | 52.78 |
| Test | | 9.87 | 8.93 | 11.21 | 9.77 | |
| formulation | Female | 231.82 ± | 250.73 ± | 276.83 ± | 287.65 ± | 55.83 |
| | | 8.99 | 9.10 | 11.04 | 10.94 | |

### Effect of Test Formulation on Water Intake in High Fat Diet Induced Obesity Rat Model

| | | | | | | Comp. |
|---|---|---|---|---|---|---|
| | | Water intake (ml)/day | | | | Initial vs |
| | | | After 1 | After 3 | After 6 | after 6 |
| Groups | Sex | Initial | month | months | months | months |
| Normal | Male | 27 ± | 26 ± | 28 ± | 28 ± | P > 0.05 |
| control | | 4.19 | 3.85 | 4.01 | 3.13 | |
| | Female | 24 ± | 25 ± | 25 ± | 26 ± | P > 0.05 |
| | | 3.82 | 3.11 | 4.12 | 3.15 | |
| High Fat Diet | Male | 26 ± | 28 ± | 27 ± | 27 ± | P > 0.05 |
| | | 3.08 | 31 | 2.95 | 4.01 | |
| | Female | 25 ± | 27 ± | 27 ± | 26 ± | P > 0.05 |
| | | 2.91 | 3.01 | 2.93 | 3.11 | |
| High Fat Diet + | Male | 26 ± | 26 ± | 28 ± | 27 ± | P > 0.05 |
| Test | | 4.01 | 3.25 | 3.11 | 3.95 | |
| formulation | Female | 24 ± | 26 ± | 25 ± | 27 ± | P > 0.05 |
| | | 4.10 | 3.22 | 3.14 | 2.50 | |

### Changes in Feed Intake Following Test Formulation Treatment in High Fat Diet Induced Obesity Rat Model

| | | | | | | Comp. |
|---|---|---|---|---|---|---|
| | | Feed intake (g)/day | | | | Initial vs |
| | | | After 1 | After 3 | After 6 | after 6 |
| Groups | Sex | Initial | month | months | months | months |
| Normal | Male | 16 ± | 17 ± | 19 ± | 18 ± | P > 0.05 |
| control | | 2.33 | 4.01 | 2.28 | 3.13 | |
| | Female | 15 ± | 16 ± | 16 ± | 17 ± | P > 0.05 |
| | | 2.11 | 1.93 | 2.13 | 1.85 | |
| High Fat Diet | Male | 15 ± | 17 ± | 20 ± | 20 ± | P > 0.05 |
| | | 2.83 | 2.65 | 2.4 | 3.02 | |
| | Female | 16 ± | 18 ± | 18 ± | 19 ± | P > 0.05 |
| | | 1.26 | 1.94 | 2.25 | 3.15 | |
| High Fat Diet + | Male | 16 ± | 16 ± | 17 ± | 16 ± | P > 0.05 |
| Test | | 3.05 | 3.01 | 2.28 | 1.36 | |
| formulation | Female | 17 ± | 18 ± | 17 ± | 17 ± | P > 0.05 |
| | | 2.14 | 1.96 | 2.12 | 2.06 | |

### Effect of Test Formulation on Biochemical Parameters in High Fat Diet Induced Obesity Rat Model

| | | Biochemical parameters | | | | |
|---|---|---|---|---|---|---|
| | | Glucose | Cholesterol | TG | Protein | Albumin |
| Groups | Sex | (mg/dl) | (mg/dl) | (mg/dl) | (g/dl) | (g/dl) |
| Normal | Male | 129 ± 6.8 | 114 ± 16.2 | 83 ± 11.6 | 9.8 ± 0.34 | 4.3 ± 0.28 |
| control* | Female | 102 ± 7.2 | 121 ± 9.8 | 88 ± 10.5 | 9.1 ± 0.82 | 4.1 ± 0.73 |
| High Fat Diet** | Male | 138 ± 20.2 | 186 ± 40.2 | 141 ± 24.2 | 9.0 ± 0.68 | 3.8 ± 0.85 |
| | Female | 136 ± 23.9 | 169 ± 31.6 | 128 ± 6.8 | 8.7 ± 1.02 | 3.7 ± 0.73 |
| High Fat Diet + | Male | 132 ± 8.1 | 159 ± 17.6 | 119 ± 9.8 | 9.4 ± 0.59 | 3.9 ± 0.31 |
| Test | Female | 127 ± 24.9 | 142 ± 18.8 | 109 ± 14.2 | 8.9 ± 0.72 | 4.0 ± 0.45 |
| formulation*** | | | | | | |

| | | | | | | |
|---|---|---|---|---|---|---|
| Comp. | | | | | | |
| *vs** | (Male) | P > 0.05 | P > 0.05 | P < 0.01 | P > 0.05 | P > 0.05 |
| *vs** | (Female | P > 0.05 | P > 0.05 | P < 0.01 | P > 0.05 | P > 0.05 |
| *vs*** | (Male) | P < 0.01 | P > 0.05 | P > 0.05 | P > 0.05 | P > 0.05 |
| *vs*** | (Female) | P > 0.05 | P > 0.05 | P > 0.05 | P > 0.05 | P > 0.05 |
| **vs*** | (Male) | P > 0.05 | P > 0.05 | P > 0.05 | P > 0.05 | P > 0.05 |
| **vs*** | (Female) | P > 0.05 | P > 0.05 | P > 0.05 | P > 0.05 | P > 0.05 |

### Beneficial Effect of Test Formulation in Reducing Leptin and Adiponectin Enhancing Effect in High Fat Diet Induced Obesity Rat Model

| | | | | | | | | | Comp. |
|---|---|---|---|---|---|---|---|---|---|
| | | | | | Comp. | | | | Initial |
| | | Leptin (ng/ml) | | | Initial | Adiponectin (μg/ml) | | | vs. |
| | | | After 1 | After 6 | vs. After 6 | | After 1 | After 6 | After 6 |
| Groups | Sex | Initial | month | months | months | Initial | month | months | months |
| Normal | Male | 1.41 ± 0.39 | 1.53 ± 0.67 | 1.48 ± 0.82 | P > 0.05 | 18.90 ± 2.45 | 15.22 ± 3.10 | 19.18 ± 3.16 | P > 0.05 |
| control | Female | 1.52 ± 0.60 | 1.35 ± 0.28 | 1.42 ± 0.45 | P > 0.05 | 16.45 ± 4.87 | 15.96 ± 3.89 | 17.34 ± 2.64 | P > 0.05 |
| High Fat | Male | — | 26.95 ± 5.11 | 28.32 ± 4.23 | P < 0.001 | — | 8.93 ± 2.86 | 7.11 ± 1.93 | P < 0.001 |
| Diet | Female | — | 27.84 ± 4.72 | 28.78 ± 5.11 | P < 0.001 | — | 9.39 ± 2.25 | 8.81 ± 2.64 | P > 0.05 |
| High Fat | Male | — | 18.95 ± 2.87 | 16.38 ± 3.11 | P < 0.001 | — | 13.57 ± 2.45 | 14.95 ± 2.90 | P > 0.05 |
| Diet + Test | Female | — | 16.48 ± 2.43 | 17.95 ± 2.28 | P < 0.001 | — | 12.32 ± 2.13 | 14.02 ± 3.16 | P > 0.05 |
| formulation | | | | | | | | | |

### Effect of Test Formulation on Insulin Level in High Fat Induced Obesity Rat Model

| | | | | | Comp. |
|---|---|---|---|---|---|
| | | Insulin (μIU/mL) | | | Initial vs |
| | | | After 1 | After 6 | After 6 |
| Groups | Sex | Initial | month | months | months |
| Normal | Male | 24.19 ± | 23.78 ± | 25.13 ± | P > 0.05 |
| control | | 3.87 | 4.10 | 4.06 | |
| | Female | 23.74 ± | 25.11 ± | 23.96 ± | P > 0.05 |
| | | 2.95 | 3.14 | 4.01 | |
| High Fat Diet | Male | — | 15.82 ± | 14.79 ± | P < 0.001 |
| | | | 2.45 | 2.04 | |
| | Female | — | 16.04 ± | 15.93 ± | P < 0.001 |
| | | | 1.91 | 2.14 | |
| High Fat Diet + | Male | — | 17.65 ± | 20.25 ± | P < 0.001 |
| Test | | | 4.81 | 3.88 | |
| formulation | Female | — | 18.94 ± | 21.82 ± | P < 0.001 |
| | | | 2.48 | 4.09 | |

### Clinical Evidence:

TABLE 1
Effect of test formulation on body mass index in obese cases
(FIG. 2)

| | | | | | | Comp. |
|---|---|---|---|---|---|---|
| | | | BMI | | | Initial vs |
| | | | | After 3 | After 6 | after 6 |
| | | No. of | | months | months | months |
| Groups | Sex | Cases | Initial | Therapy | Therapy | therapy |
| Treated with | Male | 65 | 34.71 ± 3.25 | 32.89 ± 3.01 | 30.62 ± 2.71 | P < 0.001 |
| Fluoxetine | Female | 82 | 36.39 ± 4.72 | 34.09 ± 3.42 | 31.82 ± 3.94 | P < 0.001 |
| 20 mg/day | | | | | | |
| Treated with | Male | 68 | 35.88 ± 3.91 | 33.65 ± 2.48 | 32.94 ± 2.86 | P < 0.001 |
| Test | Female | 88 | 37.11 ± 3.82 | 36.32 ± 2.59 | 34.73 ± 3.01 | P < 0.001 |
| Formulation | | | | | | |

Normal range: 18-24

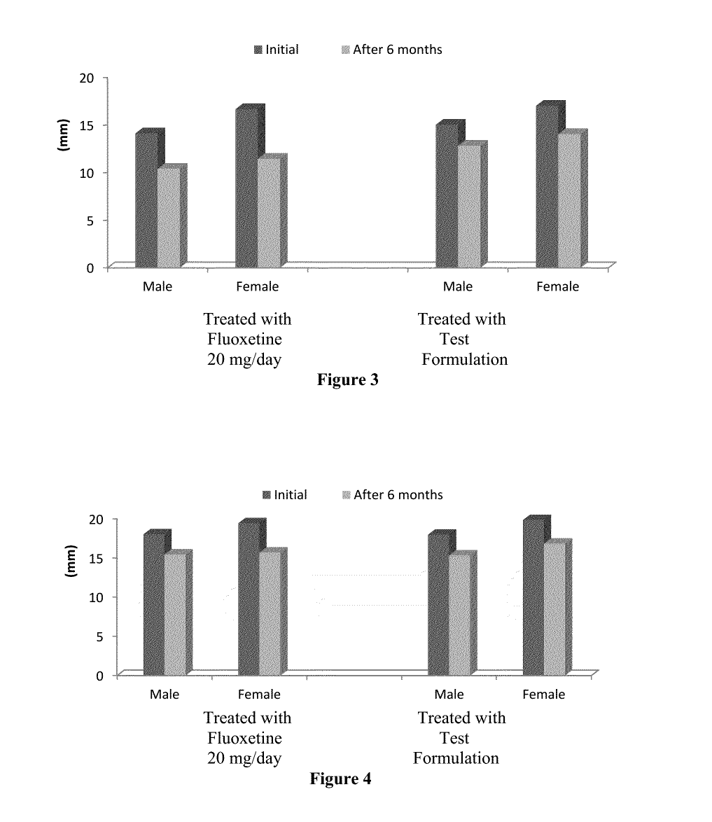

TABLE 2
Effect of test formulation on obesity index measured in terms of
Tricep Skin fold Thickness in obesity cases (FIG. 3)

| | | | Triceps Skin fold thickness | | | Comp. |
|---|---|---|---|---|---|---|
| | | | (mm) | | | Initial vs |
| | | | | After 3 | After 6 | after 6 |
| | | No. of | | months | months | months |
| Groups | Sex | Cases | Initial | Therapy | Therapy | therapy |
| Treated with | Male | 65 | 14.11 ± 2.14 | 12.62 ± 1.98 | 10.41 ± 2.02 | P < 0.001 |
| Fluoxetine | Female | 82 | 16.68 ± 3.01 | 14.34 ± 2.76 | 11.45 ± 2.97 | P < 0.001 |
| 20 mg/day | | | | | | |
| Treated with | Male | 68 | 15.01 ± 3.21 | 13.62 ± 3.05 | 12.82 ± 2.25 | P < 0.001 |
| Test | Female | 88 | 17.03 ± 3.19 | 16.48 ± 3.11 | 14.03 ± 2.81 | P < 0.001 |
| Formulation | | | | | | |

TABLE 3
Effect of test formulation on obesity index measured in terms of
Sub-scapular Skin Fold Thickness in obese cases (FIG. 4)

| | | | | | | Comp. |
|---|---|---|---|---|---|---|
| | | | Sub-scapular (mm) | | | Initial vs |
| | | | | After 3 | After 6 | after 6 |
| | | No. of | | months | months | months |
| Groups | Sex | Cases | Initial | Therapy | Therapy | therapy |
| Treated with | Male | 65 | 18.01 ± 3.02 | 16.42 ± 2.38 | 15.43 ± 2.11 | P < 0.001 |
| Fluoxetine | Female | 82 | 19.42 ± 2.84 | 17.36 ± 2.65 | 15.68 ± 3.01 | P < 0.001 |
| 20 mg/day | | | | | | |
| Treated with | Male | 68 | 17.95 ± 3.12 | 16.48 ± 3.07 | 15.31 ± 3.02 | P < 0.001 |
| Test | Female | 88 | 19.82 ± 3.44 | 18.35 ± 3.11 | 16.81 ± 2.75 | P < 0.001 |
| Formulation | | | | | | |

The Ayurvedic test formulation has revealed significant effect on various biomarkers responsible for obesity like leptin and adiponectin. A high values of leptin and significant low values of adiponectin was the main laboratory finding among the selected obesity cases. It is observed that Ayurvedic test drug treatment reduces the leptin level and simultaneously, significant increase in adiponectin level indicated the anti-obesity property of test formulation. On comparison when the initial values are compared with 6 months values of the parameters, the changes are significant. It has been observed that the results are comparable with conventional treated group (Table 4, 5).

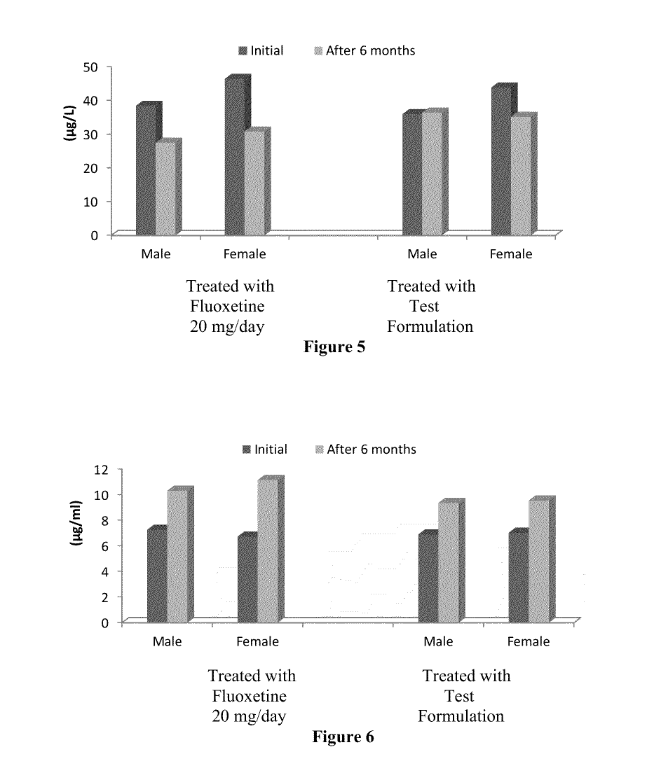

TABLE 4
Effect of test formulation on Leptin levels among obese cases
(FIG. 5)

| | | | | | | Comp. |
|---|---|---|---|---|---|---|
| | | | Leptin (μg/L) | | | Initial vs |
| | | | | After 3 | After 6 | after 6 |
| | | No. of | | months | months | months |
| Groups | Sex | Cases | Initial | Therapy | Therapy | therapy |
| Treated with | Male | 65 | 38.42 ± 6.92 | 31.69 ± 8.42 | 27.44 ± 10.06 | P < 0.001 |
| Fluoxetine | Female | 82 | 46.40 ± 9.92 | 38.11 ± 10.36 | 30.72 ± 7.90 | P < 0.001 |
| 20 mg/day | | | | | | |
| Treated with | Male | 68 | 35.96 ± 10.01 | 30.42 ± 7.38 | 26.42 ± 8.11 | P < 0.001 |
| Test | Female | 88 | 43.87 ± 12.16 | 38.30 ± 8.95 | 35.16 ± 6.84 | P < 0.001 |
| Formulation | | | | | | |

Normal range: 5-15 μg/L

TABLE 5
Effect of test formulation on Adiponectin levels among obese
cases

| | | | Adiponectin (μg/mL) | | | Comp. Initial |
|---|---|---|---|---|---|---|
| | | | | After 3 | After 6 | vs after 6 |
| | | No. of | | months | months | months |
| Groups | Sex | Cases | Initial | Therapy | Therapy | therapy |
| Treated with | Male | 65 | 7.24 ± 0.82 | 8.90 ± 0.75 | 10.31 ± 0.87 | P < 0.001 |
| Fluoxetine | Female | 82 | 6.71 ± 0.64 | 9.62 ± 0.82 | 11.14 ± 0.98 | P < 0.001 |
| 20 mg/day | | | | | | |
| Treated with | Male | 68 | 6.88 ± 0.56 | 7.69 ± 0.95 | 9.35 ± 1.03 | P < 0.001 |
| Test | Female | 88 | 7.01 ± 0.91 | 8.66 ± 0.85 | 9.54 ± 1.11 | P < 0.001 |
| Formulation | | | | | | |

Normal range: 5-30 μg/mL

High triglycerides content was also estimated in those obese cases. Maximum cases showed triglycerides more than 300 mg/dl before starting the treatment. During 6 months study period the level declined in both the groups but reduction is more in Ayurvedic test formulation treated group (Table 6).

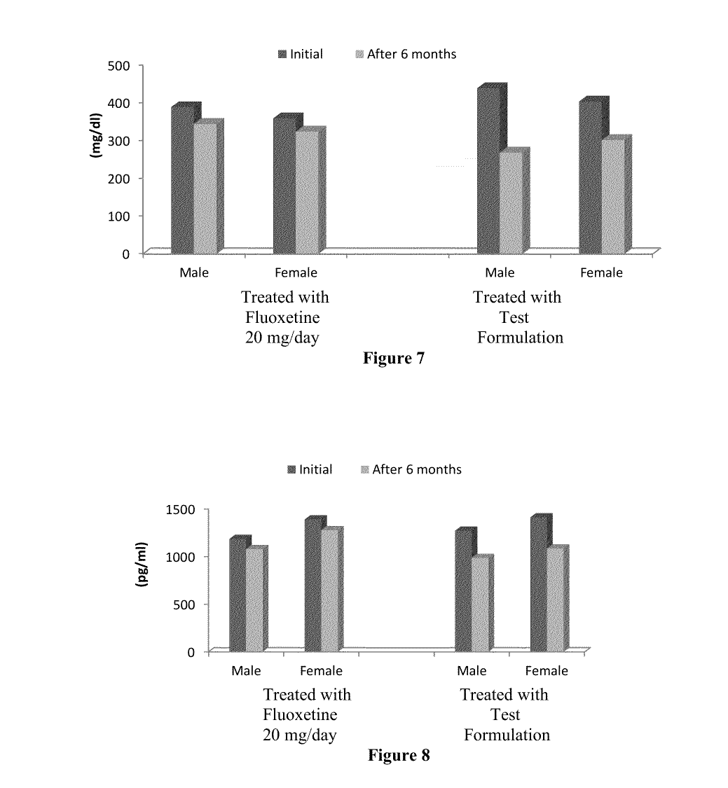

TABLE 6
Decrease in Triglycerides level following test drug treatment
among obese cases (FIG. 7)

| | | | Triglyceride (mg/dl) | | | Comp. Initial |
|---|---|---|---|---|---|---|
| | | | | After 3 | After 6 | vs after 6 |
| | | No. of | | months | months | months |
| Groups | Sex | Cases | Initial | Therapy | Therapy | therapy |
| Treated with | Male | 65 | 388.73 ± 52.84 | 352.84 ± 92.64 | 343.86 ± 65.35 | P < 0.01 |
| Fluoxetine | Female | 82 | 358.42 ± 43.72 | 341.96 ± 76.01 | 323.88 ± 53.87 | P < 0.001 |
| 20 mg/day | | | | | | |
| Treated with | Male | 68 | 438.96 ± 62.80 | 335.90 ± 51.72 | 268.94 ± 40.91 | P < 0.001 |
| Test | Female | 88 | 403.82 ± 58.99 | 351.83 ± 61.30 | 301.77 ± 52.61 | P < 0.001 |
| Formulation | | | | | | |

Normal range : ≦150 mg/dl

The increased level of endotheline and Apolipo B at initial stage indicated the risk of CHD among those cases due to endothelial dysfunction. It is observed that both endotheline and Apolipo B reduced drastically during study period (Table 7, 8). It is observed that the mode of action of present Ayurvedic formulation is through the regulation of leptin and adiponectin and thus reduces the adiposity, body fat and insulin resistance among obese individuals.

TABLE 7
Reduction in Endotheline following test drug treatment among
obese cases (FIG. 8)

| | | | Endothelin (pg/ml) | | | Comp. Initial |
|---|---|---|---|---|---|---|
| | | | | After 3 | After 6 | vs after 6 |
| | | No. of | | months | months | months |
| Groups | Sex | Cases | Initial | Therapy | Therapy | therapy |
| Treated with | Male | 65 | 1185.73 ± 238.64 | 1125.32 ± 198.40 | 1078.91 ± 260.00 | P < 0.02 |
| Fluoxetine | Female | 82 | 1390.15 ± 248.64 | 1309.46 ± 208.62 | 1277.42 ± 190.84 | P < 0.01 |
| 20 mg/day | | | | | | |
| Treated with | Male | 68 | 1271.62 ± 190.75 | 1038.65 ± 213.44 | 985.76 ± 128.84 | P < 0.001 |
| Test | Female | 88 | 1411.73 ± 210.88 | 1238.60 ± 168.40 | 1084.62 ± 108.35 | P < 0.001 |
| Formulation | | | | | | |

Normal range: 0.32-1000 pg/ml

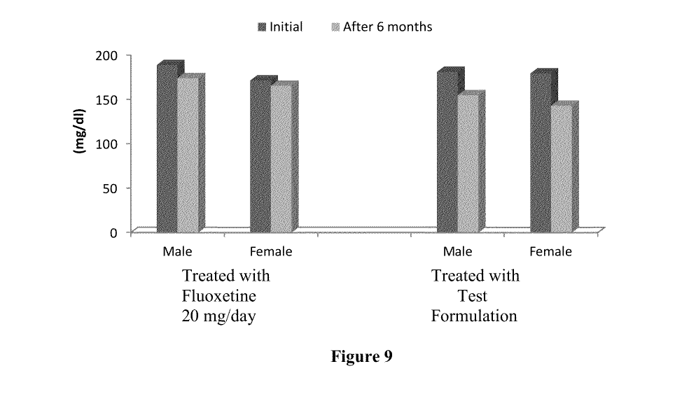

TABLE 8
Reduction in Apolipo B content following test drug treatment
among obese cases (FIG. 9)

| | | | Apolipo (B) (mg/dl) | | | Comp. Initial |
|---|---|---|---|---|---|---|
| | | | | After 3 | After 6 | vs after 6 |
| | | No. of | | months | months | months |
| Groups | Sex | Cases | Initial | Therapy | Therapy | therapy |
| Treated with | Male | 65 | 188.71 ± 41.75 | 180.69 ± 38.59 | 173.94 ± 45.33 | P > 0.05 |
| Fluoxetine | Female | 82 | 171.32 ± 38.02 | 160.82 ± 36.92 | 165.44 ± 41.06 | P > 0.05 |
| 20 mg/day | | | | | | |
| Treated with | Male | 68 | 181.06 ± 28.41 | 170.44 ± 30.41 | 154.82 ± 26.55 | P < 0.001 |
| Test | Female | 88 | 179.31 ± 51.12 | 162.75 ± 47.30 | 142.89 ± 38.21 | P < 0.001 |
| Formulation | | | | | | |

Normal range: 55-159 mg/dl

### REFERENCE

1. Abenhain L, Moride Y, Brenot F, et al N Engl J Med, 335:609-16; 1996.
2. Allison, D. B, Fontaine, K. R, Manson, J E, Stevens, J., Vanitallie, T. B, Annual deaths attributable to obesity in the united states. JAMA 282, 1530-1538; 1999
3. Allison L. Diamant, Susan H. Babey, Joelle W. and Malia J.: Obesity and Diabetes: Two growing epidemics in California. UCLA Centre for Health Policy Research, 2010.
4. American Diabetes Association “Type 2 Diabetes” Http://www.diabetes.org/type-2-diabetes.jsp., February, 2004.
5. American Diabetes Association “Type 2 Diabetes” The metabolic syndrome” http://www.diabetes.org/weightlossandexercise/weightloss/metabolicsyndrome.jsp; February, 2004.
6. American obesity Association. AOA lact sheets: “Health effects of obesity” http:/www.obesity.org/subs/fastfacts/health%20-effects.html; February, 2004.
7. Arita Y, Kihara S, Ouchi N, Takahashi M, Maeda K, Miyagawa J, Hotta K, Shimomura I, Nakamura T, Miyaoka K, Kuriyama H, Nishida M, Yamashita S, Okubo K, Matsubara K, Muraguchi M, Ohmoto Y, Funahashi T, Matsuzawa Y: Paradoxical decrease of an adipose-specific protein, adiponectin, in obesity. Biochem Biophys Res Commun 257:79-83, 1999.
8. Aronne, L. J. Treatment of obesity in the primary case setting Wadden, T. Strenbard, A. J. eds, Handbook of obesity treatment, 383-394 Guiyord Press New York; 2002.
9. Barzilay J I, Abraham L, Heckbert S R, et al. The relation of markers of inflammation to the development of glucose disorders in the elderly: the Cardiovascular Health Study. Diabetes. 50:2384-2389; 2001.
10. Bender R, Jockel K H, Trautner C, Spraul M, Merger M. Effect of age on excess mortality in obesity JAMA. 281:1498-1504; 1999.
11. Bouchard C, Bray G, Hubbard V S. Am J Clin Nutr, 52:946-50, 1990.
12. Bowen R, Glicklich A, Kahn M, et al. Cardiac valulopathy associated with exposure to fenfluramine or dexfenfluramine: U.S. Department of Health and human Services Interim Public Health Recommendations. Morbidity Mortality Weekly report. 46:1061-6, 1997.
13. Calle E E, Thun M J, Petrelli J M, Rodriguez C, Health C W. Jr. Body-mass index and mortality in a prospective cohort of U.S. adults. N. Engl J Med. 341: 1097-1105; 1999.
14. Chessler S D, Fujimoto W Y, Shofer J B, et al. Increased plasma leptin levels are associated with fat accumulation in Japanese Americans. Diabetes. 47: 239-243; 1998.
15. De Simone G, Mancini M, Mainenti G, Turco S, Ferrara L A. Weight reduction lowers blood pressure independently of salt restriction. J Endocrinol Invest. 15:339-343; 1992.
16. Engeland A, Bjorge T, Sogaard A J, Tverdal A. Body mass index in adolescence in relation to total mortality: 32-year follow-up of 227,000 Norwegian boys and girls. Am J Epidemiol. 157:517-23; 2003.
17. Flagal, K. M., Carroll, M. D., Ogden, C L, Johnson, C L. Prevalence and trends in obesity among U.S. adults, 1999-2000. JAMA 2888, 1723-1727; 2002.
18. Freeman D J, Norrie J, Caslake M J, et al. C-reactive protein is an independent predictor of risk for the development of diabetes in the West of Scotland Coronary Prevention Study. Diabetes. 51:1596-1600; 2002.
19. Graddock D. Obesity and its management. 3rd ed. Edinburgh: Churchill Livingstone, 92-109; 1978.
20. troop LC, Saloranta C, Shank M, Bonadonna R C, Ferrannini E, DeFronzo R A. The role of free fatty acid metabolism in the pathogenesis of insulin resistance in obesity and noninsulin-dependent diabetes mellitus. J Clin Endocrinol Metab. 72:96-107; 1991.
21. Han S H, Quon M J, Jeong-a Kim, Koh K K. Adiponectin and Cardiovascular Disease, J Am Coll Cardiol, 2007; 49:531-538.
22. Hill J O, Hauptman J, Anderson J W, et al Orlistat, a lipase inhibitor, for weight maintenance after conventional dieting: a 1-y study. Am J din Nutr. 69:1108-1116; 1999.
23. Hotamisligil G S, Arner P, Caro J F, Atkinson R L, Spiegelman B M. Increased adipose tissue expression of tumor necrosis factor-alpha in human obesity and insulin resistance. J Clin Invest.; 95:2409-2415; 1995.
24. Hotamisligil G S, Murray D L, Choy L N, Spiegelman B M. TNF-α inhibits signaling from the insulin receptor. Proc Natl Acad Sci USA.; 91:4854-4858, 1994.
25. Hotamisligil G S, Peraldi P, Budavari A, Ellis R, White M F, Spiegelman B M. IRS-1-mediated inhibition of insulin receptor tyrosine kinase activity in TNF-alpha- and obesity-induced insulin resistance. Science. 271:665-668; 1996.
26. Hotamisligil G S, Shargill N S, Spiegelman B M. Adipose expression of tumor necrosis factor-α: direct role in obesity-linked insulin resistance. Science. 259:87-91; 1993.
27. Hotamisligil G S, Spiegelman B M. Tumor necrosis factor α: a key component of the obesity-diabetes link. Diabetes. 43:1271-1278; 1994.
28. Huang Z, Hankinson S E, Colditz G A et at Dual effects of weight and weight gain on breast cancer risk. JAMA, 278:1407-1411; 1997.
29. Joint National Committee on Prevention, Evaluation, and Treatment of High Blood Pressure. The Sixth Report of the Joint national Committee on Prevention, Detection, Evaluation, and Treatment of High Blood pressure. Bethesda, Md.: National Institute of Health; National Heart, Lung and Blood Institute; 1997.
30. Lapidus L, Bengtsson C, Larsson B, et al Br. Med J, 289: 1257-61; 1984.
31. McNeely M J, Boyko E J, Weigle D S, et al. Association between baseline plasma leptin levels and subsequent development of diabetes in Japanese Americans. Diabetes Care. 22: 65-70; 1999.
32. Mohamed-Ali V, Pinkney J H, Coppack S W. Adipose tissue as an endocrine and paracrine organ. Int J Obes Relat Metab Disord. 22:1145-58; 1998.
33. Mokdad, A. et al., Actual causes of Death in the United states, 2000. JAMA, 29:1238-1245; 2004.
34. Narkiewicz, K, Somers, V K, Mos, L, et al. An independent relationship between plasma leptin and heart rate in untreated patients with essential hypertension J Hypertens 17, 245-249; 1999.
35. National Institute of Health: National Institute of Diabetes, Digestive and Kidney Diseases “statistics related to overweight and obesity: The economic costs” http:/www.win.niddk.nih.gov/statistics/index.htm: December, 2004
36. National Task Force on the Prevention and Treatment of obesity “Overweight, obesity and health risk” Archives of Internal Medicine, 2000, 160: 898-904
37. North American Association for study of obesity “your weight and diabetes” http://www.naaso.org/information/diabetesobesity.asp, February, 2004
38. Popkin B M, Drewnowksi A. Dietary fats and the nutrition transition: new trends in the global diet. Nutr Rev. 55:31-43; 1997.
39. Pradhan A D, Manson J E, Rifai N, Buring J E, Ridker P M. C-reactive protein, interleukin 6, and risk of developing type 2 diabetes mellitus. JAMA. 286:327-334; 2001.
40. Rolls B J, Shide D J, Thorwart M L, Ulbrecht J S. Sibutramine reduces food intake in non-dieting women with obesity. Obes Res. 6:1-11; 1998.
41. Rosengren A, Wedel H, Wilhelmsen L. Body weight and weight gain during adult life in men in relation to coronary heart disease and mortality. A prospective population study. Eur Heart J. 20:269-77; 1999.
42. Rossner S, Bjorvell H. Early and late effects of weight loss on lipoprotein metabolism in severe obesity. Atherosclerosis. 64:125-130; 1987.
43. Scherrer V, Nussberger J, Torriani S, Waeber B, Dariolo R, Hofstetter J R, Brunner H R. Effect of weight reduction in moderately overweight patients on recorded ambulatory blood pressure and free cytosolic platelet calcium. Circulation. 83:552-558; 1991.
44. Shoff S M, Newcomb P A. Diabetes, body size, and risk of endometrial cancer Am J Epidemiol. 148:234-240; 1998.
45. Steppan C M, Bailey S T, Bhat S, et al. The hormone resistin links obesity to diabetes. Nature.; 409:307-312, 2001.
46. Weyer C, Yudkin J S, Stehouwer C D, Schalkwijk C G, Pratley R E, Tataranni P A: Humoral markers of inflammation and endothelial dysfunction in relation to adiposity and in vivo insulin action in Pima Indians. Atherosclerosis 161:233-242, 2002
47. World Health Organization. Obesity: preventing and management the global epidemic. Report of a WHO consultation presented at: the World Health Organization; Jun. 3-5, 1997; Geneva, Switzerland. Publication WHO/NUT/NCD/98.1.
48. Yarnell J W, Patterson C C, Thomas H F, Sweetnam P M. Comparison of weight in middle age, weight at 18 years, and weight change between, in predicting subsequent 14 year mortality and coronary events: Caerphilly Prospective study. J Epidemiol Community Health. 54:344-8; 2000.
49. Yudkin J S, Kumari M, Humphries S E, et al. Inflammation, obesity, stress and coronary heart disease: is interleukin-6 the link? Atherosclerosis; 148: 209-14; 2000.
50. Zimmet P Z, Collins V R, de Courten M P, et al. Is there a relationship between leptin and insulin sensitivity independent of obesity? A population-based study in the Indian Ocean nation of Mauritius: Mauritius NCD Study Group. Int J Obes. 22: 171-177; 1998.
51. Yang W S, Lee W J, Funahashi T, Tanaka S, Matsuzawa Y, Chao C L, Chen C L, Tai T Y, Chuang L M: Weight reduction increases plasma levels of an adipose-derived anti-inflammatory protein, adiponectin. J Clin Endocrinol Metab 86:3815-3819, 2001
52. Dubey G. P., Agrawal A., Rajamanickam G. V, Lavekar G. S. Anti-oxidant property of Hippophae rhamnoides and its role in the management of neurodegenerative disorders. Book: Brain Ageing and Ayurveda, published by Central Council for Research in Ayurveda and Siddha, Dept. of AYUSH, Ministry of Health and Family Welfare, Govt. of India, New Delhi, 2008.
53. Sauvaire Y, Ribes G, Baccou J C, et al Implication of steroid saponins and sapogenins in the hypocholesterolemic effect of fenugreek, Lipids. 26(3): 191-197, 1991.
54. Reddy D. B., Reddy I C M, Jyotsna G, Satish Saran, Nalini Priya, Laxmipati V, N. Reddanna P.: Chebulagic acid a COX-LOX dual inhibitor isolated from the fruits of Terminalia chebula. Retz, induces apoptosis in COLO-205 Cell line Pub. Journal of Ethanopharmacology, Vol. 124, Issue 3 pp. 506-512, 2009.
55. Singh I, Singh P. K., Bhansali S., Shafiqu N, Malhotra S, Pandhi P, Singh A P: effect of 2 different doses of a fruit extract of Terminalia chebula on metabolic components of metabolic syndrome in a rat model. Pub. Phyto-therapy research 2009.
56. Matsuda, H., Murakami, T., Yashiro, K., Yamahara J., Yoshikawa, M., Anti-diabetic principles of natural medicine. IV Aldose reductase and a-glucosidas inhibitors from the roots of Salacia reticulata Wall. (Celastraceae): structure of a new friedelane-type triterpene kotalagenin 16-acetate. Chemical & Pharmaceutical Bulletin 47, 1725-1729; 1999.
57. Matsuda. H., Morikawa. T., Yoshikawa, M., Anti-diabetogenic constituents from several natural medicines. Pure and Applied Chemistry 74, 130-1308; 2002.
58. Shimoda. H., Kawamori. S., Kawahara, Y, Effects of an aqueous extract of Salacia reticulata, a useful plant in Sri Lanka, on postprandial hyperglycemia in rats and humans., Nippon Eiyo Shoukuryo Gakkaishi 151, 279-287; 1998.
59. Mishra A K, Mrinal M, J. Bapat, Tilkal & Thomas, Devasagayam P A.: Antioxidant activity of Garcinia indica fruits syrup. Current Sciences Vol. 91, No. 1 2006.
60. Jena, B. S., Jayaprakasha, G. K., Singh, R. P. and Sakariah, K. K., Chemistry and biochemistry of (−)-hydroxycitric acid from Garcinia. J. Agric. Food Chem., 50, 10-22; 2002.
61. Amol Bhalchandra Deore, Vinayak Dnyandev Sapakal, Neelam Laxman Dashputre and Nilofer S. Naikwade: Antiulcer activity of Garcinia indica linn fruit rinds: Journal of Applied Pharmaceutical Science 01 (05); 151-154, 2011.
62. K. N. Varalakshmi, C. G. Sangeetha, A. N. Shabeena, S. R. Sunitha and J. Vapika. Antimicrobial and Cytotoxic Effects of Garcinia indica Fruit Rind Extract. American Eurasian J. Agric. & Environ. Sci., 7 (6): 652-656, 2010.
63. Fumio Yamaguchi, Toshiaki Ariga, Yoshihiro Yoshimura, and Hiroyuki Nakazawa. Antioxidative and Anti-Glycation Activity of Garcinol from Garcinia indica Fruit Rind: J. Agric. Food Chem., 2000, 48 (2), pp 180-185
64. Tusharendra Singh, S. B. Kasture P. K. Mohanty Yusuf Jaliwala Manvendra Singh Karchuli Abhishek Agarwal and Yashraj Yadav: Cyclophosphamide-induced oxidative stress in brain: Protective effect of Garcinia indica fruit extract: INTERNATIONAL JOURNAL OF PHARMACY & LIFE SCIENCES: September, 2011.

All documents cited in the description are incorporated herein by reference. The present invention is not to be limited in scope by the specific embodiments and examples which are intended as illustration of a number of aspects of the scope of this invention. Those skilled in the art will know or to be able to ascertain using no more than routine experimentation many equivalents to the specific embodiments of the invention described herein.

It is to be further noted that present invention is susceptible to modifications adoptions and changes by those skilled in the art. Such variant embodiments employing the concepts and features of this invention are intended to be within the scope of the present invention which is further set forth under the claims:—

1. A plant based formulation for the management of obesity comprising an effective amount of hydro-alcoholic extracts of Salacia reticulata, Garcinia indica, Dioscorea bulbifera, Terminalia chebula and optional additives.

2. The plant based formulation as claimed in claim 1 wherein the said hydro-alcoholic extract is water: alcohol in the ratio of 50:50.

3. The plant based formulation as claimed in claim 1 wherein the additives are selected from minerals, vitamins, salts and binders.

4. The plant based formulation as claimed in claim 1 wherein the plant extracts are from the plant parts:

| Name of the plants | Part used |
|---|---|
| 1. Salacia reticulata (Saptachakra) | Root |
| 2. Garcinia indica (Kokum) | Fruit |
| 3. Dioscorea bulbifera (Varahikand) | Rhizome |
| 4. Terminalia chebula (Harilaki) | Fruits and Seeds |

5. The plant based formulation as claimed in claim 1 wherein the plant extracts are present in following effective amounts:

| Name of the plants | Amount |
|---|---|
| 1. Salacia reticulata | 200-450 mg |
| 2. Garcinia indica | 100-300 mg |
| 3. Dioscorea bulbifera | 150-425 mg |
| 4. Terminalia chebula | 150-350 mg |

6. The plant based formulation as claimed in claim 1 wherein the effective amount of plant extracts of Dioscorea bulbifera and Salacia reticulata has lipid lowering activity and has anti-atherosclerotic role.

7. The plant based formulation as claimed in claim 1 wherein the effective amount of plant extracts of Dioscorea bulbifera, Terminalia chebula and Garcinia indica has anti-hyperlipidemic activity.

8. A plant based formulation for management of obesity, comprising:
an effective amount of a hydro-alcoholic extract of at least two plants selected from the group consisting of Salacia reticulata, Garcinia indica, Dioscorea bulbifera, and Terminalia chebula.

9. The formulation of claim 8, further comprising at least one additive selected from the group consisting of minerals, vitamins, salts, and binders.

10. The formulation of claim 8, wherein the plant extracts are from the plant parts:

| Name of the plants | Part used |
|---|---|
| 1. Salacia reticulata (Saptachakra) | Root |
| 2. Garcinia indica (Kokum) | Fruit |
| 3. Dioscorea bulbifera (Varahikand) | Rhizome |
| 4. Terminalia chebula (Harilaki) | Fruits and Seeds |

11. The formulation of claim 8, wherein the plant extracts are present in an amount to provide doses of:

| Name of the plants | Amount |
|---|---|
| 1. Salacia reticulata | 200-450 mg/day |
| 2. Garcinia indica | 100-300 mg/day |
| 3. Dioscorea bulbifera | 150-425 mg/day |
| 4. Terminalia chebula | 150-350 mg/day |

12. The formulation of claim 8, comprising hydro-alcoholic extracts of Salacia reticulata and Dioscorea bulbifera.

13. The formulation of claim 8, comprising hydro-alcoholic extracts of Garcinia indica, Dioscorea bulbifera, and Terminalia chebula.

14. The formulation of claim 8, comprising hydro-alcoholic extracts of Salacia reticulata, Garcinia indica, and Terminalia chebula.

15. The formulation of claim 8, comprising hydro-alcoholic extracts of Salacia reticulata, Dioscorea bulbifera, and Terminalia chebula.

16. The formula of claim 8, wherein the formula is in the form of a pharmaceutical capsule or tablet.

17. A capsule or tablet for the management of obesity comprising a plant based formulation that comprises therapeutically effective amounts of Salacia reticulata, Garcinia indica, Dioscorea bulbifera, and Terminalia chebula.

18. The capsule or tablet of claim 17, wherein the plant based formulation consists essentially of Salacia reticulata, Garcinia indica, Dioscorea bulbifera, and Terminalia chebula.
